(19) United States
(12) Patent Application Publication
(10) Pub. No.: US 2015/0307833 A1
(43) Pub. Date: Oct. 29, 2015

(54) IDENTIFICATION OF THERMOTOLERANCE GENES IN YEAST

(71) Applicants: VIB VZW, Gent (BE); KATHOLIEKE UNIVERSITEIT LEUVEN, K.U.LEUVEN R&D, Leuven (BE)

(72) Inventors: Johan Thevelein, Blanden (BE); Maria Remedios Foulquie-Moreno, Brussel (BE); Yudi Yang, Leuven (BE)

(21) Appl. No.: 14/651,610

(22) PCT Filed: Dec. 12, 2013
(86) PCT No.: PCT/EP2013/076344

§ 371 Date: Jun. 11, 2015

(30) Foreign Application Priority Data
Dec. 12, 2012 (EP) 12196785.5

(51) Int. Cl. C12N 1/18; C07K 14/47; C12Q 1/68

(52) U.S. Cl. CPC C12N 1/18; C12Q 1/6895; C07K 14/47; C12Q 2600/156; C12Q 2600/16

(57) ABSTRACT

The disclosure relates to a method for identifying genes that are important determinators of thermotolerance in yeast. The disclosure relates further to genes, identified with the method, especially specific alleles of PRP42 and the use of such alleles to increase thermotolerance. It relates further to recombinant strains transformed with such alleles.

### CROSS-REFERENCE TO RELATED APPLICATIONS

This application is a national phase entry under 35 U.S.C. §371 of International Patent Application PCT/EP2013/076344, filed Dec. 12, 2013, designating the United States of America and published in English as International Patent Publication WO 2014/090930 A1 on Jun. 19, 2014, which claims the benefit under Article 8 of the Patent Cooperation Treaty and under 35 U.S.C. §119(e) to European Serial No. 12196785.5, filed Dec. 12, 2012.

### TECHNICAL FIELD

The disclosure relates to a method for identifying genes that are important determinators of thermotolerance in yeast. The disclosure relates further to genes, identified with the method, especially specific alleles of PRP42 and/or SMD2 and the use of such alleles to increase thermotolerance. It relates further to recombinant strains transformed with such alleles.

### BACKGROUND

Many genetic traits are quantitative and show complex inheritance. Because these traits are so prevalent in nature, understanding the underlying factors is important for various biological fields and applications including agriculture, industrial biotechnology and human disease (Paterson 1998). The study of quantitative traits has its origin in agricultural practice, in which commercially-important features of plants and animals are improved by breeding (Lynch and Walsh 1998). Over the last two decades, much attention has been paid to identifying quantitative trait loci and/or genes contributing to genetic diversity in human disease susceptibility and severity (Risch 2000), since most human diseases seem to be caused by multiple genes instead of single genes (Botstein and Risch 2003). Baker's yeast Saccharomyces cerevisiae has become an important subject for studies in quantitative genetics during the last ten years for several reasons. First, it has a fully sequenced (Goffeau et al., 1996) and well-annotated genome, which enables easy establishment of high-density genetic markers. Second, the ease of making experimental crosses and the relatively high frequency of recombination events in meiosis facilitate identification of QTLs and subsequent fine-mapping (Mancera et al., 2008). Third, the efficiency of genetic engineering strongly simplifies the identification and detailed interaction analysis of causative genes. Fourth, S. cerevisiae displays many quantitative traits that are also important in other cell types, including industrial microorganisms and cells of higher, multicellular organisms. Such properties include thermotolerance (Steinmetz et al., 2002) and oxidative stress tolerance (Diezmann and Dietrich 2011), two factors required for making S. cerevisiae, an opportunistic pathogen, the capacity to produce small molecules such as acetic acid (Marullo et al., 2007), ethanol tolerance (Hu et al., 2007; Swinnen et al., 2012) and many other important features for industrial microorganisms. Other quantitative traits that have been studied in yeast include transcriptional regulation (Brem et al., 2002), sporulation efficiency (Ben-Ari et al., 2006), telomere length (Gatbonton et al., 2006), cell morphology traits (Nogami et al., 2007), mitochondrial genome instability (Dimitrov et al., 2009), global gene expression (Ehrenreich et al., 2009), evolution of biochemical pathways (Bullard et al., 2010) and resistance to chemicals (Ehrenreich et al., 2010).

### BRIEF SUMMARY

Despite the recent surge in quantitative trait studies with S. cerevisiae, several issues still remain to be solved. A major challenge is the efficient mapping of minor quantitative trait loci (QTLs) and identification of their causative genes. Minor QTLs have a subtle influence on the phenotype, which is easily masked by epistasis (Carlborg and Haley 2004), gene-environment interactions (Smith and Kruglyak 2008), low association to the phenotype because of limited sample size and complex interactions with QTLs that act in a redundant way. Mainly two methods have been reported to address this problem. Sinha et al., 2008, used a targeted backcross strategy to eliminate the superior allele that plays a major role in the phenotype. A subsequent mapping study enabled the authors to reveal a novel allele that had an epistatic interaction with the previously identified major gene. A disadvantage of this approach is the reduction of genetic diversity in the backcrossing, which easily leads to loss of minor QTLs. In addition, this approach effectively reveals minor QTLs that independently produce additional effects, but may miss QTLs with an epistatic interaction with the major QTL that was eliminated. An important disadvantage of elimination of major QTLs in one parent is the reduction in phenotypic difference between the two parent strains, which makes phenotypic screening much more cumbersome. In a second approach, Swinnen et al., 2012, made use of more stringent phenotyping, tolerance to 17% versus 16% ethanol, which effectively revealed several additional minor QTLs. The disadvantage of this approach is that higher stringency of phenotyping requires much higher numbers of segregants to be phenotyped in order to obtain enough segregants displaying the superior phenotype.

In this work we have established a novel approach for identifying minor QTLs that is based on the observation that superior haploid segregants of heterozygous natural or industrial diploid strains with a superior phenotype often contain recessive mutations that to some extent compromise rather than promote the phenotype of interest. As a result genetic mapping with such segregants often reveals QTLs which are linked to the inferior parent rather than to the superior parent. This allows the construction of two new parent strains, which are both downgraded for the trait of interest by replacement of a superior allele with an inferior allele from the other parent. As a result, the new downgraded parent strains remain sufficiently different in the trait of interest for efficient QTL mapping and also retain all their genetic diversity, in particular all remaining minor QTLs and causative genes. We show the effectiveness of this approach by first mapping QTLs involved in high thermotolerance of a selected yeast strain compared to a control strain, identifying causative genes linked to the superior and inferior parent, constructing two downgraded parent strains and repeating the genetic mapping with the downgraded parent strains. This revealed several new minor QTLs of which we have validated two by identifying the causative gene. Interestingly, the two novel causative genes identified in this study are both involved in pre-mRNA splicing, which suggests an important role for RNA processing in conferring high thermotolerance.

A first aspect of the disclosure is the use of a small nuclear ribonucleoprotein particle protein to obtain thermotolerance in yeast. Preferably, the use is the use of a Prp42 protein and/or a Smd2 protein to obtain thermotolerance in yeast. PRP42 is known to the person skilled in the art and encodes an essential protein for U1 small nuclear ribonucleoprotein (snRNP) biogenesis, which has a high similarity to Prp39 (McLean and Rymond 1998). Similarly, SMD2 is known to the person skilled in the art, and the gene is also encoding a small ribonucleoprotein particle protein. Preferably, the Prp42 protein as well as the Smd2 protein are encoded by a specific allele (indicated as “superior allele”), even more preferably by the Prp42 protein is encoded by the allele as present in strain BY4742 and the Smd2 protein is encoded by the allele as present in strain 21A. More preferably, the Prp42 protein comprises, even more preferably consists of SEQ ID NO:2 and the Smp2 protein comprises, even more preferably consists of SEQ ID NO:4. In one preferred embodiment, the use is the replacement of an endogeneous allele by the superior allele. A preferred embodiment is the replacement by the superior allele of PRP42. Another preferred embodiment is the replacement with the superior allele of SMD2. Still another preferred embodiment is the replacement with both the superior alleles of both PRP42 and SMD2. On another preferred embodiment, the use is overexpression of the gene, preferably of the superior allele, encoding the Prp42 protein and/or the overexpression of the gene, preferably the superior allele, encoding the Smd2 protein. Overexpression, as used herein, means that the expression of the gene in the modified strain is higher than in the parental strain, when grown under the same conditions. As a non-limiting example, overexpression can be obtained by increasing the copy number of the gene, or by replacing the endogeneous promoter by a stronger promoter. Preferably, the yeast is a Saccharomyces sp., even more preferably a Saccharomyces cerevisiae. Thermotolerance, as used herein, means that the yeast can grow at high temperatures, preferably at a temperature of more than 40° C., even more preferably more than 40.5° C., most preferably at 40.7° C. or higher.

Another aspect of the disclosure is a recombinant yeast, preferably a Saccharomyces sp., even more preferably a Saccharomyces cerevisiae, comprising a recombinant gene encoding a Prp42 protein, preferably a protein comprising, even more preferably consisting of SEQ ID NO:2. Preferably, the gene comprises SEQ ID NO:1 and/or comprising a recombinant gene encoding a Smd42 protein, preferably a protein comprising, even more preferably consisting of SEQ ID NO:4. Preferably, the gene comprises SEQ ID NO:3.

Still another aspect of the disclosure is a method to obtain a thermotolerant yeast, comprising the crossing of two parental strains, wherein at least one parental strain encodes a protein comprising SEQ ID NO:2 and/or a protein comprising SEQ ID NO:4.

Another aspect of the disclosure is a method to obtain a thermotolerant yeast, comprising a transformation with a gene encoding a protein consisting of SEQ ID NO:2 and/or SEQ ID NO:4. Preferably, the gene comprises SEQ ID NO:1 and/or SEQ ID NO:3. In one preferred embodiment, the yeast is transformed with a gene encoding a protein consisting of SEQ ID NO:2, preferably a gene comprising SEQ ID NO:1. In another preferred embodiment, the yeast is transformed with a gene encoding a protein consisting of SEQ ID NO:4, preferably a gene comprising SEQ ID NO:3. In still another preferred embodiment, the yeast is transformed with both the genes encoding the protein consisting of SEQ ID NO:2 and SEQ ID NO:4, preferably both the gene comprising SEQ ID NO:1 and the gene comprising SEQ ID NO:3.

Still another aspect of the disclosure is a method for screening thermotolerance in yeast, comprising (1) identifying at least one gene responsible for thermotolerance (2) downgrading the gene in a yeast strain (3) crossing two downgraded strains (4) screening for thermotolerance genes in the offspring of the cross. “Downgrading,” as used herein, means that an allele conferring thermotolerance is replaced by an allele conferring lower thermotolerance, as can be determined by growing the two strains comprising the two different alleles at a critical temperature. Preferably, at least two genes responsible for thermotolerance are identified, and each gene is downgraded in one of the parental strains used in the cross.

### BRIEF DESCRIPTION OF THE FIGURES

FIG. 1. Thermotolerance of the parent strains and segregants. The diploid strain MUCL28177 was identified as a highly thermotolerant strain, showing strong growth at 41° C. One of its haploid segregants MUCL28177-21A (referred to as 21A) showed comparably high thermotolerance, whereas the control laboratory strain BY4742 does not grow at all at 41° C. The hybrid diploid strain 21A/BY4742 grows as well at 41° C. as its superior parent 21A, indicating that the major causative allele(s) in 21A is (are) dominant. The haploid segregants from 21A/BY4742 show varying growth ability at 41° C., between that of the BY4742 inferior and 21A superior parents, indicating that thermotolerance is a quantitative trait.

FIG. 2. Genetic mapping of QTLs involved in thermotolerance by pooled-segregant whole-genome sequence analysis. Genomic DNA samples were extracted from two pools of thermotolerant segregants. Pool 1 contained 58 thermotolerant segregants, able to grow at 41° C., from the cross between the original parents 21A and BY4742. Pool 2 contained 58 thermotolerant segregants able to grow at 40.7° C., from the cross between the downgraded parents 21A^{DG} and BY4742^{DG}. Genomic DNA of the thermotolerant parent 21A and the two thermotolerant segregant pools, was sequenced and the reads aligned to the reference genome, S288c (isogenic to BY4742), to identify SNPs. The SNP variant frequency of quality-selected SNPs, displaying high coverage and high abundance, from pool 1, derived from the original parents, (small circles) and pool 2, derived from the downgraded parents, (small plusses) was plotted against the SNP chromosomal position to identify putative QTLs. Smoothened lines were obtained for pool 1 (green line) and pool 2 (red line) using a generalized linear mixed model. Dashed lines indicate confidence intervals. The lower panel shows the corresponding 2-sided P-value calculation based on the predicted SNP variant frequency. QTL1-7, identified in the mapping with the original parents (pool 1) (broken black lines), and QTL8-10, identified in the mapping with the downgraded parents (pool 2) (stippled black lines), are indicated at the corresponding positions.

FIG. 3. Dissection of QTL1 to identify the causative gene. (A) Fine-mapping of QTL1 by scoring selected SNPs in the individual thermotolerant segregants. The region between 400,000 bp and 550,000 bp in chromosome XIV showed the strongest linkage to the 21A parent genome among all QTLs. Eight SNPs spanning this region were scored by PCR in 46 thermotolerant segregants and both SNP variant frequency and FDR P-value were calculated. A 60,000 bp region between SNP2 and 5 showed the strongest linkage. It contained 33 genes and putative ORFs as indicated using the annotations in SGD. The genes containing at least one non-synonymous mutation within the ORF are indicated with an asterisk. (B) Reciprocal hemizygosity analysis (RHA) for individual genes. For each candidate gene, two diploid 21A/BY4742 strains were constructed, with one hybrid containing only one, BY4742 derived allele, and the other only one, 21A derived allele. (C) Identification of the causative gene MKT1 in QTL1. RHA results for all individual genes in the central region of QTL1 are shown. The strain pairs for the same genes were always spotted on the same plate. The results for the original hybrid diploid 21A/BY4742 and the MKT1 reciprocal deletion strains were also from the same plate.

FIG. 4. Dissection of QTL3 to identify the causative gene. (A) Fine-mapping of QTL3 by scoring seven selected SNPs in 62 individual thermotolerant segregants confirms significant linkage with the genome of the inferior parent strain BY4742 of the region between 930,000 and 970,000 bp on chromosome IV. This region contained the indicated 25 genes and putative ORFs as annotated in SGD. The genes containing at least one non-synonymous mutation within the ORF are indicated with an asterisk. To facilitate identification of the causative genes, this region has been divided into two fragments for bulk RHA, as indicated. (B) Example of bulk RHA for the block of genes on FRAGMENT1. A pair of reciprocal deletion strains for either FRAGMENT1 or FRAGMENT2 was constructed as shown and tested for growth at high temperature. (C) Identification of the causative gene in QTL3. Bulk RHA shows that FRAGMENT1, derived from BY4742, confers higher thermotolerance compared to FRAGMENT1, derived from 21A, whereas for FRAGMENT2 there was no difference. RHA for the individual genes within FRAGMENT1 identified PRP42 as the causative gene. It was the only gene causing a significant difference on thermotolerance for the two alleles, with the PRP42-BY4742 allele from the inferior parent as the superior allele.

FIG. 5. Comparison of thermotolerance in the FRAGMENT1 and PRP42 reciprocal deletion strains. All strains were spotted on the same plate and incubated at 41° C.

FIG. 6. Thermotolerance of the downgraded parent strains and their segregants. (A) Growth at 41° C. of the original parent strains, 21A and BY4742, the downgraded parent strains, 21A^{DG} and BY4742^{DG}, and hybrid diploids in the four combinations. All strains were spotted on the same plate. (B) Growth at 40.7° C. of the original parent strains, 21A and BY4742, the downgraded parent strains, 21A^{DG} and BY4742^{DG}, and ten segregants from the hybrid 21A^{DG}/BY4742^{DG}. The strain pairs for the same genes were always spotted on the same plate.

FIG. 7. RHA for NCS2 as candidate causative gene in the new QTL8 identified with the downgraded parents. NCS2-21A conferred higher thermotolerance than NCS2-BY4742, confirming NCS2 as causative gene in QTL8. Deletion of NCS2-BY4742 in 21A^{DG}/BY4742^{DG} also reduced thermotolerance.

FIG. 8. Dissection of the new QTL9 identified with the downgraded parents to identify the causative gene. (A) Fine mapping of QTL9 by scoring six selected SNPs in 58 individual thermotolerant segregants confirm significant linkage of the region between 625,000 and 780,000 bp on chromosome XII to the genome of the superior 21A^{DG} parent strain. The region between 680,000 and 720,500 bp, showing the strongest linkage, was analyzed for causative gene(s). It contained the indicated genes and putative ORFs as annotated in SGD. The genes containing at least one non-synonymous mutation within the ORF are indicated with an asterisk. This region was divided into three fragments for bulk RHA, as indicated. Overlapping genes in successive fragments (SMD2 and NNT1) are indicated with a stippled box. (B) Bulk RHA with the three fragments. FRAGMENT1 and FRAGMENT2 from the 21A^{DG} parent strain confer higher thermotolerance at 40.7° C. than the corresponding fragment from BY4742^{DG}, while for FRAGMENT3 there was no difference. RHA for the individual genes within FRAGMENT1 and FRAGMENT2 showed that only for the gene SMD2 in the overlap between the two fragments there was a difference in thermotolerance. The SMD2-21A allele conferred higher thermotolerance compared to the SMD2-BY4742 allele. (C) RHA for SMD2 in the hybrid strain made with the original parents, 21A/BY4742, failed to reveal any difference in thermotolerance either at 40.7 or 41° C. conferred by the two SMD2 alleles.

FIG. 9. SNP variant frequency for the unselected segregants pool. 58 segregants from the hybrid, 21A^{DG}/BY4742^{DG}, made with the downgraded parent strains, were randomly chosen. Their DNA was isolated, sequenced, and the data processed in the same way as for the two selected pools. The SNP variant frequency was plotted against the SNP chromosomal position (small x symbols) and the smoothened line, calculated as for the selected pools, is shown in blue. The dashed lines indicate confidence intervals.

FIG. 10. Assay of thermotolerance in strains with different MKT1 alleles. The superior 21A strain with either mkt1Δ or MKT1-BY4742 (21ADG) shows the same growth at 40.7° C. The BY4742 strain shows the same growth at 40.7° C. as the BY4742 mkt1Δ strain. This indicates that the MKT1-BY4742 allele behaves as a loss of function allele for thermotolerance when assayed under these conditions and in these haploid strain backgrounds.

### DETAILED DESCRIPTION

### Examples

### Materials and Methods to the Examples

Yeast Strains, Growth Conditions and Sporulation

Yeast cells were grown in YPD medium containing 1% (w/v) yeast extract, 2% (w/v) bacteriological peptone, and 2% (w/v) glucose. 1.5% (w/v) Bacto agar was used to make solid nutrient plates. Transformants were grown on YPD agar plates containing 200 μg/ml geneticin. Mating, sporulation and isolation of haploid segregants were done using standard protocols (Sherman and Hicks 1991).

Phenotyping

Strains were inoculated in liquid YPD and grown in a shaking incubator at 30° C. overnight. The next day the cells were transferred to fresh liquid YPD at an OD600 of 1 and grown for 2 to 4 h to enter exponential phase. The cell cultures were then diluted to an OD600 of 0.5 μl and 5 μl of a fourfold dilution range was spotted on YPD agar plates, which were incubated at different temperatures. Growth was scored after two days incubation for all conditions. All spot tests were repeated at least once, starting with freshly inoculated cultures.

Pooled-Segregant Whole-Genome Sequence Analysis and Determination of SNP Variant Frequency

For each genetic mapping experiment, 58 thermotolerant segregants were grown separately in 50 ml liquid YPD cultures at 30° C. for three days. Cell dry weight was measured for each culture and the cultures were pooled based on the same dry weight. Genomic samples of the pooled culture, together with that of 21A were isolated with standard methods (Johnston 1994). At least 5 μg of each DNA sample was provided to GATC Biotech AG or BGI for sequencing. Paired-end short reads of 100 bp were generated. Sequence alignment was performed using SeqMan NGen. SNP calling, filtering, and frequency prediction was performed using previously described methods (Swinnen et al., 2012).

SNP Scoring in Individual Segregants

SNPs were scored in individual segregants by PCR. At a given chromosomal location, two SNPs spacing between 500 bp and 1,500 bp were chosen for the design of specific primers. For a given SNP, two primers either in the forward or reverse direction, were designed with one mismatch at their 3′ ends. First, a gradient PCR was applied using genomic samples of 21A and BY4742 as templates, with each template tested with two primer combinations (primer pair based on the sequence of BY4742 and primer pair based on the sequence of 21A). The annealing temperature at which the best distinguishing power was obtained with the two parents was used for scoring of the SNPs in the individual segregants.

Reciprocal Hemizygosity Analysis

All the ORFs of non-essential genes in the center of the QTL were deleted separately in both 21A and BY4742. PCR-mediated gene disruption was used (Winzeler et al., 1999). Plasmid pFA6a was used as a template to amplify a linear DNA fragment containing the kanMX4 cassette (Wach et al., 1994), with 50 bp homologous sequences for the target regions at both ends. Transformants growing on YPD geneticin plates were verified by PCR with several combinations of internal and external primers. The verified haploid deletion strains were subsequently crossed with the matching wild-type haploid to generate the hybrid diploids. For RHA with essential genes and fragments containing multiple genes, transformation was performed directly in the hybrid diploid. External SNPs primer pairs together with internal primers within the kanMX4 cassette were used in different combinations to determine in which parent the allele or the fragment had been deleted. For each heterozygous deletion hybrid, at least two isogenic strains were made and evaluated for thermotolerance.

Allele Replacement

The replacement of MKT1-21A with MKT1-BY4742 in 21A was performed by a two-step transformation. For the first transformation, a linear DNA fragment with the AMD1 gene from Zygosaccharomyces rouxii flanked by 50 bp sequences that are homologous to the two sides of the MKT1 ORF was amplified from plasmid pFA6a-AMD1-MX6 (Shepherd and Piper 2010) by PCR, and transformed into 21A. Transformants were grown on YCB (Yeast Carbon Base 1.17%, phosphate buffer 3%, Bacto agar 2%) plates containing 10 mM acetamide. Single colonies were checked for the correct replacement with the use of external and internal primers. For the second transformation, colonies were transformed with a linear DNA fragment containing the MKT1-BY4742 ORF, together with ˜100 bp downstream and upstream. Transformants were grown on YNB galactose (0.17 Yeast Nitrogen Base w/o amino acids and ammonium sulfate, 1.5% Difco agar, 0.01% galactose, pH 6.5) containing 100 mM fluoroacetamide. Colonies were first checked for the presence of MKT1 by PCR, and then confirmed by DNA sequencing. The replacement of PRP42-BY4742 with PRP42-21A in BY4742 was performed in a two-step transformation. For the first transformation, a URA3 gene was inserted ˜50 bp downstream of the PRP42 ORF in BY4742. Colonies growing on -URA plates were confirmed to have a correct insertion by PCR. For the second transformation, a linear DNA fragment containing the ORF of PRP42-21A together with ˜400 bp downstream and upstream was transformed into the previous colonies, and the transformants were grown on 5-FOA plates. Colonies were first checked for the right DNA polymorphism by SNP primer pairs, and then confirmed by DNA sequencing.

### Example 1

### Identification of QTLs Determining High Thermotolerance

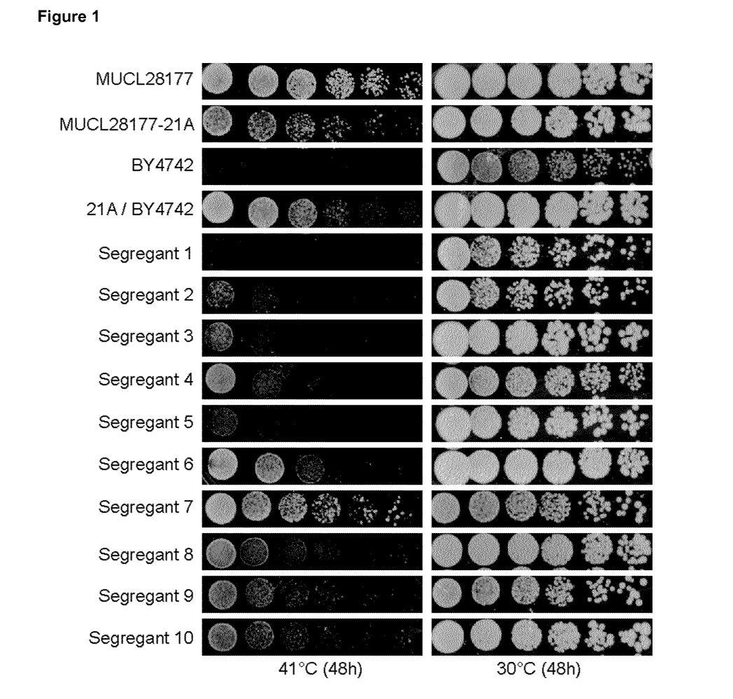

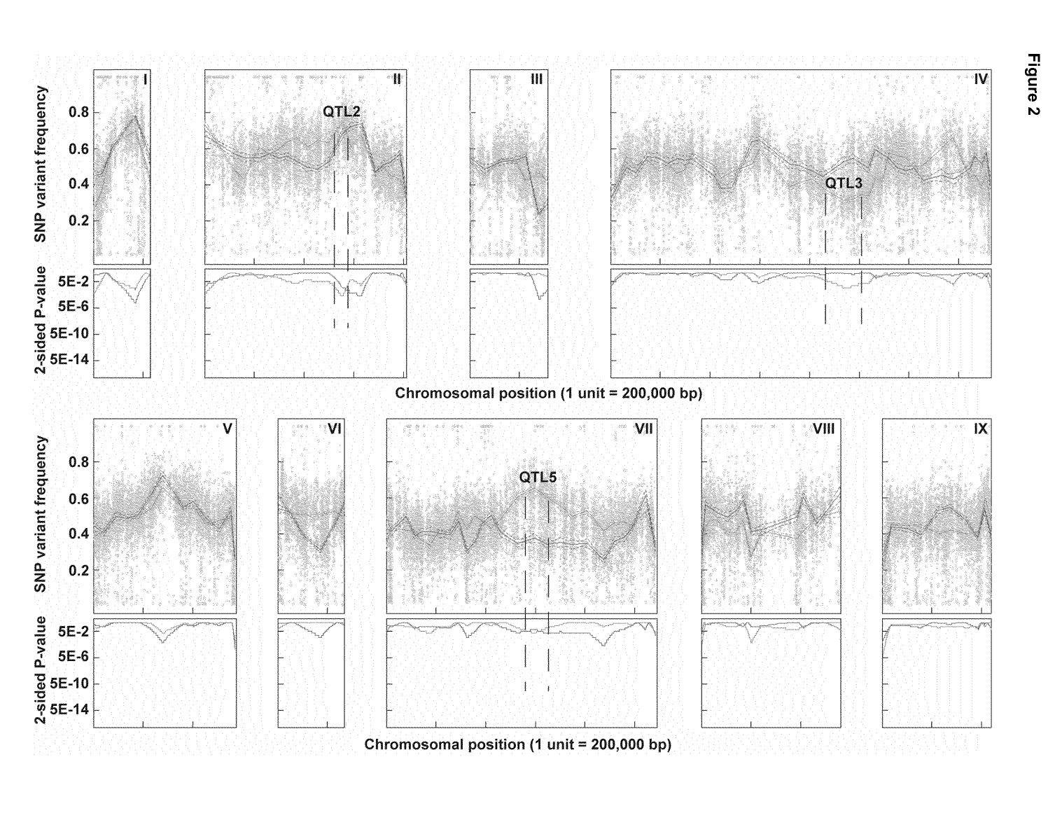

We have screened over three hundred natural and industrial isolates of S. cerevisiae for their ability to grow at high temperature, i.e., 40-41° C., on solid YPD plates. Not a single yeast strain was able to grow with a reasonable rate at 42° C. The strain MUCL28177 showed very good growth at 41° C. and was chosen for further analysis. After sporulation, we selected a haploid segregant MUCL28177-21A, further referred to as 21A, which grew almost as well as the parent strain at 41° C. Strain 21A was crossed with the laboratory strain BY4742 which is unable to grow at 41° C. The hybrid 21A/BY4742 diploid strain grew at least as well as the 21A strain at 41° C., indicating that the high thermotolerance of 21A is a dominant characteristic. Over 900 segregants of the 21A/BY4742 diploid strain were phenotyped for thermotolerance. This resulted in 58 segregants with similar growth at high temperature as 21A. The growth of the original strain MUCL28177, the parent strains 21A and BY4742, the hybrid diploid strain 21A/BY4742 and ten representative segregants with varying thermotolerance, is shown in FIG. 1.

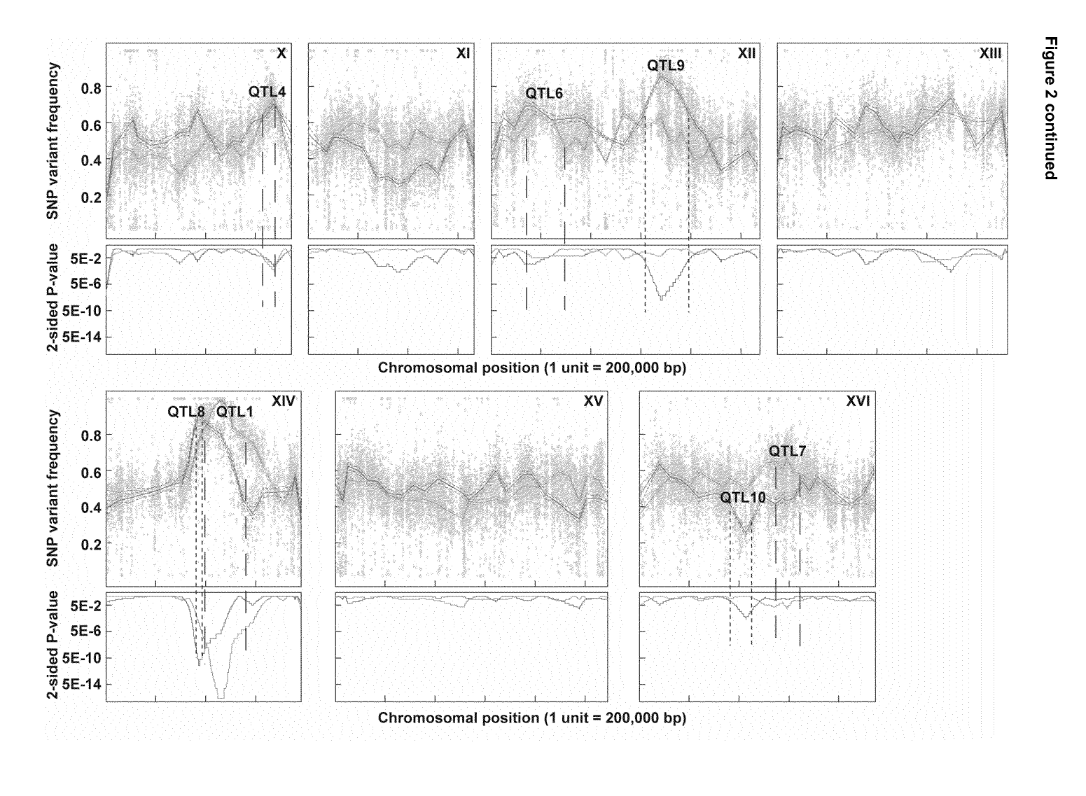

The 58 thermotolerant segregants were pooled based on dry weight and genomic DNA isolated from the pool. Genomic DNA samples from the pooled segregants and from parent strain 21A were sequenced with an average coverage of 75 and 73, respectively, by IlluminaHiSeq 2000 technology (GATC Biotech, Konstanz). The sequence reads obtained were aligned with the sequence of the reference S288c genome, which is essentially the same as that of the inferior parent strain BY4742. A set of quality-filtered SNPs to be used as genetic markers, was acquired essentially as described before (Swinnen et al., 2012). The SNP variant frequency was plotted against the chromosomal position for each chromosome and smoothened lines through the data points were calculated as described previously (Swinnen et al., 2012). The results are shown in FIG. 2. Upward deviations of the smoothened line from the mean of 0.5 indicate QTLs linked with the genome of the superior 21A parent strain, while downward deviations indicate QTLs linked with the genome of the inferior BY4742 parent strain. Normally, only linkage with the superior parent strain is expected. However, since the original MUCL28177 diploid strain is a natural isolate, it is likely heterozygous. Hence, the 21A segregant may contain recessive mutations that compromise to some extent thermotolerance in spite of the fact that its overall thermotolerance was similar to that of the MUCL28177 parent strain.

We have calculated 2-sided P-values for all SNPs (Benjamini and Yekutieli 2005) and selected for further analysis seven putative QTLs with a P-value approaching or lower than the cut-off for significance of 0.05 (FIG. 2). For all seven loci, selected SNPs were scored in individual thermotolerant segregants (up to 62 after additional segregant isolation and phenotyping) and the variant frequency for each SNP used for precise calculation of the FDR P-value (Benjamini and Yekutieli 2005; Swinnen et al., 2012). This resulted in three QTLs (QTL1, QTL2, QTL3) with a statistically significant linkage to the high thermotolerance phenotype (0.05 FDR cut-off value) (Table 1). QTL1 and QTL2 showed linkage with the genome of the superior 21A parent strain, while QTL3 showed linkage with the genome of the inferior BY4742 parent strain. Because the linkage of QTL1 was stronger than that of QTL2, we selected QTL1 and QTL3 for identification of a causative gene linked to the superior and inferior parent strain, respectively.

### Example 2

### Identification of the Causative Gene in QTL1

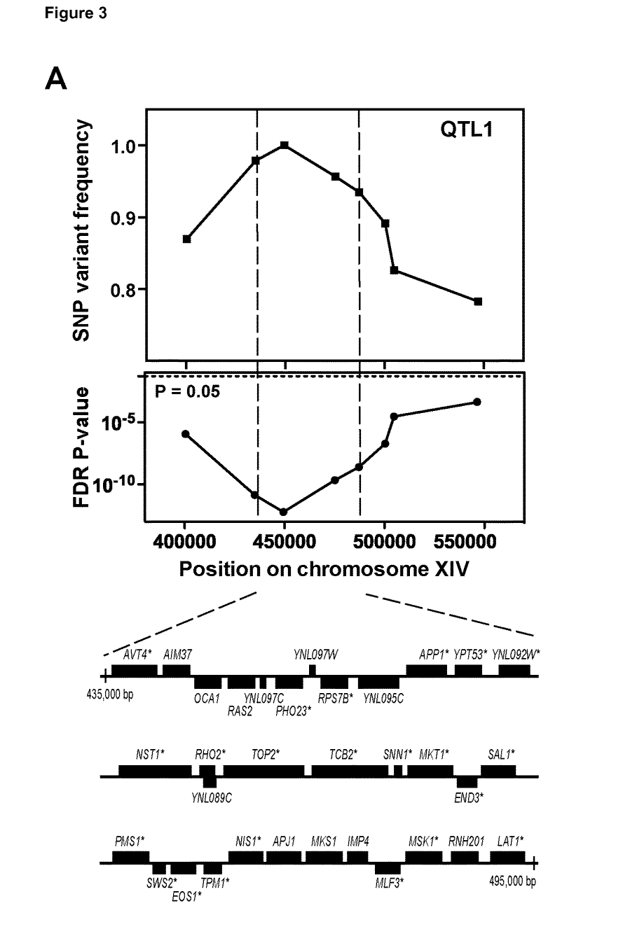

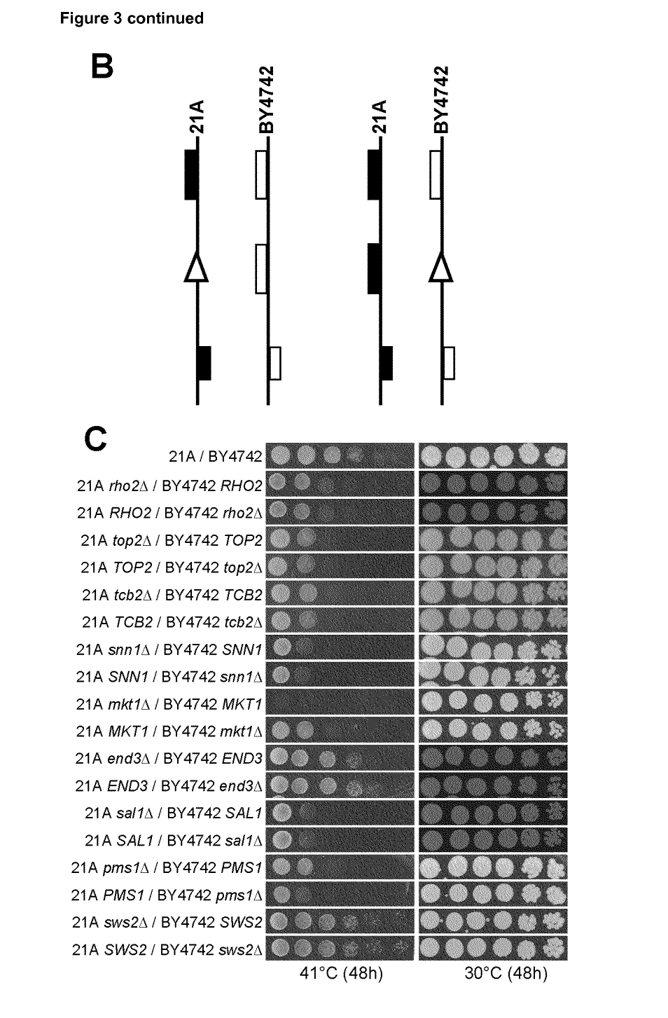

We first fine-mapped QTL1 by scoring eight selected SNPs in individual thermotolerant segregants, which reduced the size of the locus to about 60,000 bp (FIG. 3A). Detailed analysis of the 21A sequence of this region showed that 22 out of the 33 genes and putative ORFs present contained at least one non-synonymous mutation in the ORF compared to the BY4742 sequence (FIG. 3A). Next we applied reciprocal hemizygosity analysis (RHA) (Steinmetz et al., 2002) to identify causative gene(s) in QTL1. RHA is used to test for a possible contribution to the phenotype of each allele of the candidate gene in a hybrid genetic background. For each of the 22 genes with non-synonymous mutations, we constructed two 21A/BY4742 hybrid strains in which either the 21A or the BY4742 allele was deleted, so that each strain only contained one specific allele of the candidate gene (FIG. 3B). Comparison of the growth at high temperature (41° C.) of the two hybrid strains did not show any difference for the 22 candidate genes, except for MKT1 (FIG. 3C).

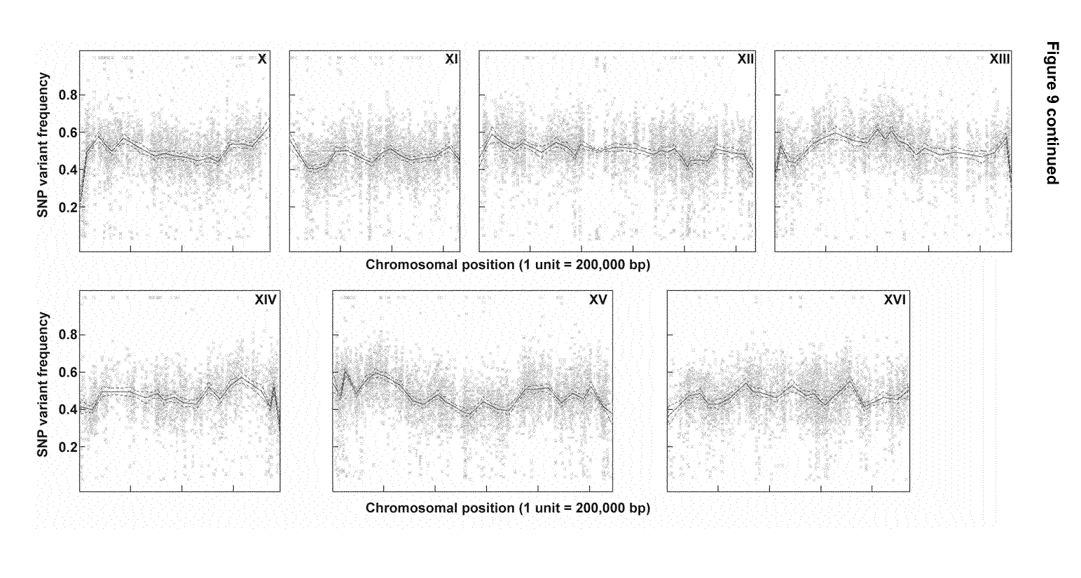

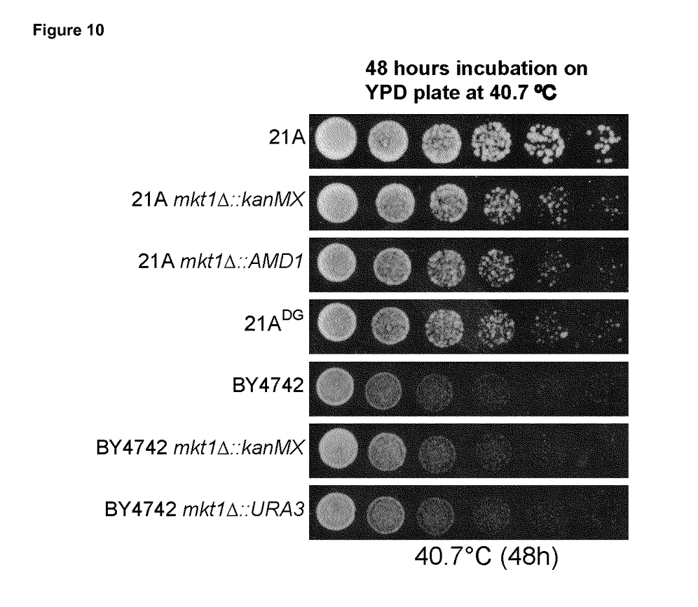

The hybrid strain with the MKT1-21A allele showed better growth than the strain with the MKT1-BY4742 allele. We further confirmed the relevance of MKT1 by demonstrating that MKT1 deletion reduced thermotolerance in the 21A strain background (FIG. 9). Since 21A with either mkt1Δ or MKT1-BY4742 showed the same growth at 40.7° C. and since BY4742 showed the same growth at 40.7° C. as BY4742 mkt1Δ, the MKT1-BY4742 allele behaves as a loss of function allele for thermotolerance when assayed under our conditions and in our haploid strain backgrounds (FIG. 9).

In a previous QTL mapping study of thermotolerance with a clinical isolate of S. cerevisiae and the lab strain S288c, the MKT1 allele of the clinical isolate was also identified as a causative gene (Steinmetz et al., 2002). In that study, END3 and RHO2, which are located closely to MKT1 in the same QTL, were also reported to have an allele-specific contribution to thermotolerance. However, in the current experimental setup, the alleles from our two genetic backgrounds did not produce a difference in thermotolerance (FIG. 3C).

### Example 3

### Identification of the Causative Gene in QTL3

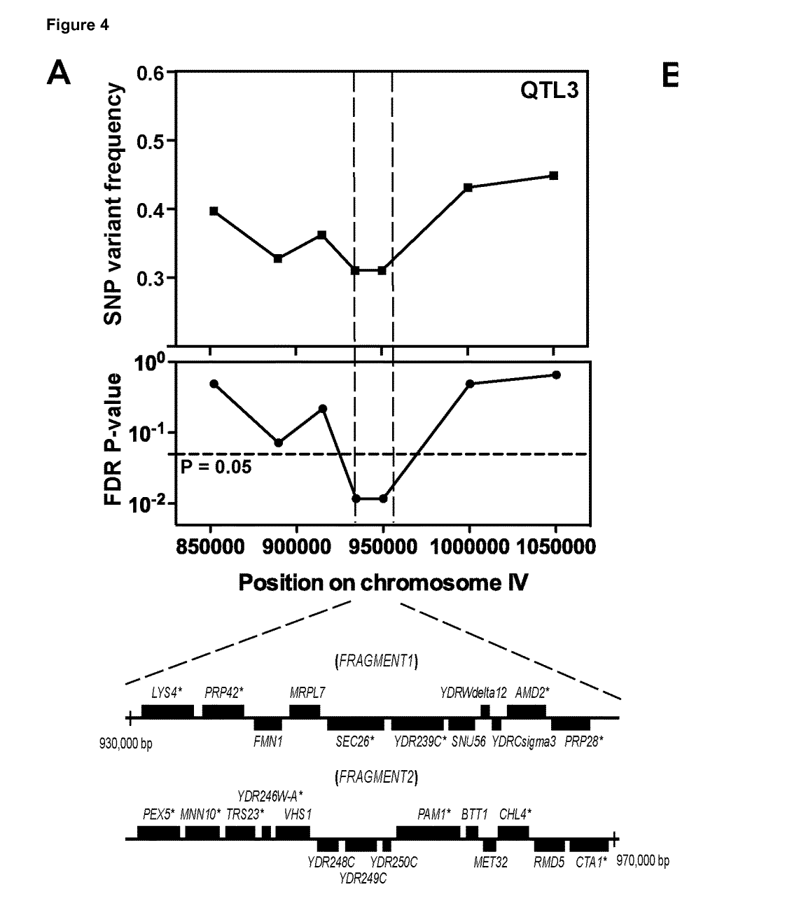

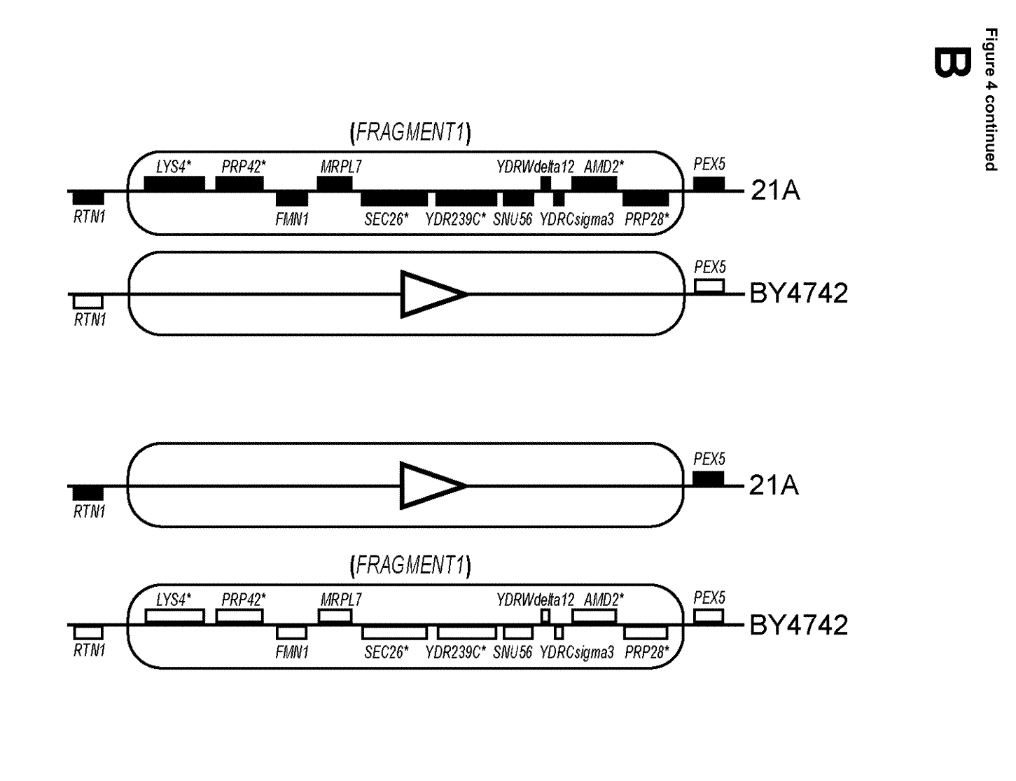

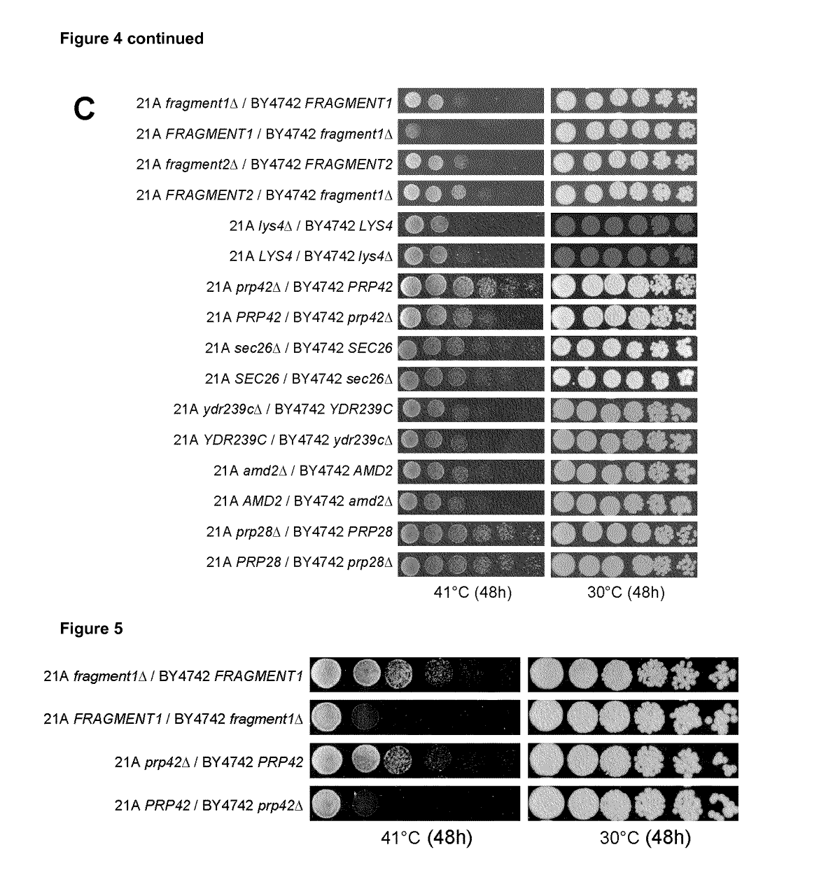

QTL3 is linked to the genome of the inferior parent strain, indicating that BY4742 contains a superior genetic element for thermotolerance in this region. This may be consistent with the observation that the hybrid 21A/BY4742 strain is growing slightly better than 21A at 41° C. (FIG. 1), although this may also be due to a genome dosage effect. We fine-mapped QTL3 by scoring seven selected SNPs in 62 thermotolerant segregants individually. This reduced the locus to 40,000 bp (FIG. 4A). Detailed analysis of the 21A sequence in this region revealed 13 genes and putative ORFs with at least one non-synonymous mutation (FIG. 4A).

To accelerate identification of the causative genes in this region, we first performed “bulk RHA.” Instead of comparing alleles for each single gene, we first made a reciprocal deletion in the hybrid strain of a fragment with multiple genes. We divided the 40,000 bp region of QTL3 into two fragments, with the first containing 11 genes and the second 14 genes (FIGS. 4A, B). For each fragment, we constructed two hybrid strains with one strain containing only the fragment from the 21A background and the other only the fragment from the BY4742 background (FIG. 4B). Comparison of growth at high temperature (41° C.) showed that FRAGMENT1-BY4742 conferred better growth at high temperature than FRAGMENT1-21A, while there was no difference between the strains with FRAGMENT2-BY4742 or FRAGMENT2-21A (FIG. 4C).

We then applied RHA for the six individual genes of FRAGMENT1 that had at least one non-synonymous mutation (FIG. 4C). This identified PRP42-BY4742 as a superior allele for thermotolerance compared to PRP42-21A, whereas for the other genes there was no allele-specific difference in thermotolerance (FIG. 4C). We also tested growth at high temperature of strains containing a heterozygous deletion of either the complete FRAGMENT1 or only the PRP42 gene together on the same plate. We found the growth at 41° C. to be virtually the same whether the complete FRAGMENT1 or only the PRP42 gene from either BY4742 or from 21A was deleted (FIG. 5). This suggests that PRP42 was likely the only causative gene in FRAGMENT1 and thus also seems to exclude the other genes without non-synonymous mutation in their ORF as possible causative gene. As an additional control, we also performed RHA with the seven genes of FRAGMENT2 with a non-synonymous mutation in their ORF and we did not find any difference between the alleles from the two parent strains in conferring thermotolerance.

After identifying MKT1-21A and PRP42-BY4742 as causative alleles for high thermotolerance, we confirmed by Sanger sequencing the identity of all SNPs in these genes. MKT1-21A contains two SNPs within the ORF that cause two protein polymorphisms in Mkt1: D30G and K453R. PRP42-BY4742 contains eleven SNPs within the ORF that cause three protein polymorphisms in Prp42: H296Y, F467S, and E526Q.

### Example 4

### Construction and Phenotyping of the Downgraded Parent Strains

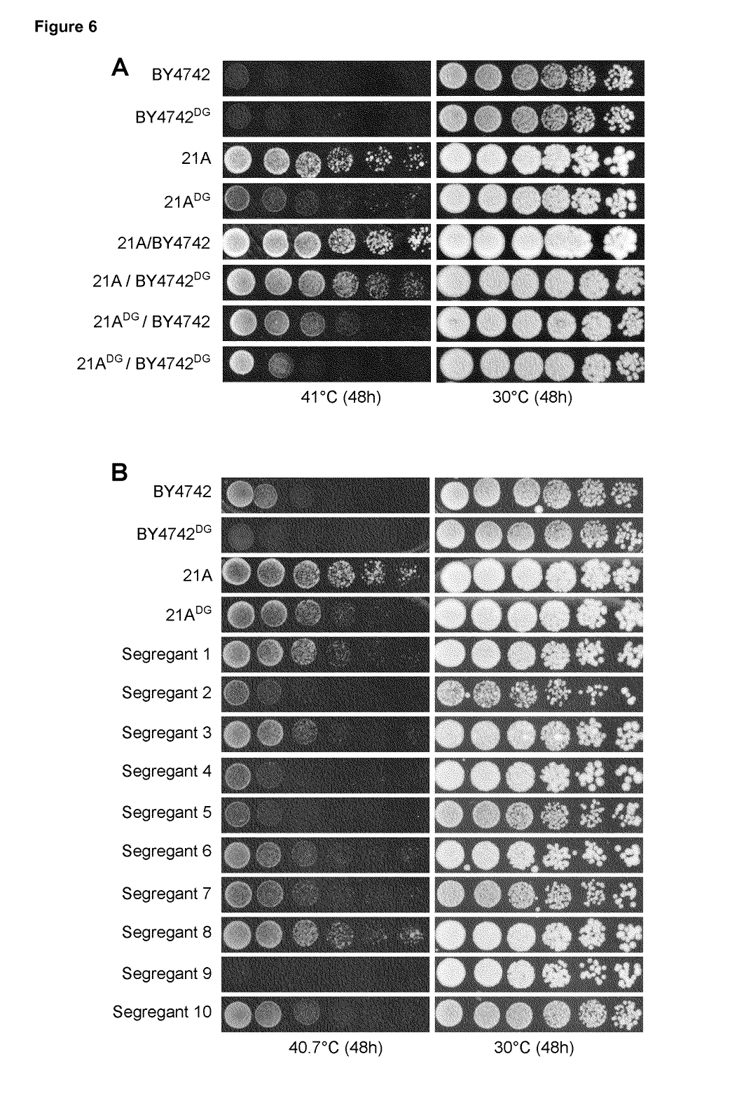

We next constructed two downgraded parent strains each with their own superior allele replaced by the inferior allele of the other parent: 21A^{DG}: 21A mkt1A::MKT1-BY4742 and BY4742^{DG}: BY4742 prp42Δ::PRP42-21A. Growth at 41° C. of 21A^{DG} was reduced compared to 21A, confirming the importance of MKT1-21A for high thermotolerance in 21A (FIG. 6A). At 41° C., BY4742 and also BY4742^{DG} are not able to grow (FIG. 6A). Hence, we reduced the temperature to 40.7° C., which allowed to demonstrate reduced growth of BY4742^{DG} compared to BY4742 (FIG. 6B). Also at 41° C., we could demonstrate the beneficial effect of PRP42-BY4742 compared to PRP42-21A by comparing growth of the 21A^{DG}/BY4742 and 21A^{DG}/BY4742^{DG} hybrid strains (FIG. 6A). The availability of the four hybrid diploid strains also allowed us to demonstrate that in this background the effect of the MKT1 and PRP42 genes on thermotolerance is additive. The hybrid diploids, 21A^{DG}/BY4742 and 21A/BY4742^{DG}, each with replacement of one superior allele, both showed reduced growth at 41° C. compared to the original hybrid of the parent strains, 21A/BY4742, while the hybrid of the two downgraded parent strains, 21A^{DG}/BY4742^{DG}, in which both superior alleles are replaced, showed further reduced growth FIG. 6A). (In this figure all strain pairs were put on the same plate.)

### Example 5

### Isolation and Phenotyping of Segregants from the Downgraded Parent Strains

FIG. 6 shows that both at 41° C. and 40.7° C., the two downgraded parent strains, 21A^{DG} and BY4742^{DG}, still show a strong difference in thermotolerance. We sporulated the 21A^{DG}/BY4742^{DG} diploid strain and phenotyped over 2,200 segregants for thermotolerance. Examples are shown in FIG. 6B. The segregants showed transgressive segregation (Rieseberg et al., 1999), since some of the segregants showed poorer thermotolerance than the inferior BY4742^{DG} parent (e.g., segregant 9 in FIG. 6B), while others showed better thermotolerance than the superior 21A^{DG} parent (e.g., segregant 8 in FIG. 6B). This suggests the presence of additional QTLs and causative genes influencing thermotolerance.

### Example 6

### Identification of New QTLs with Segregants from the Downgraded Parents

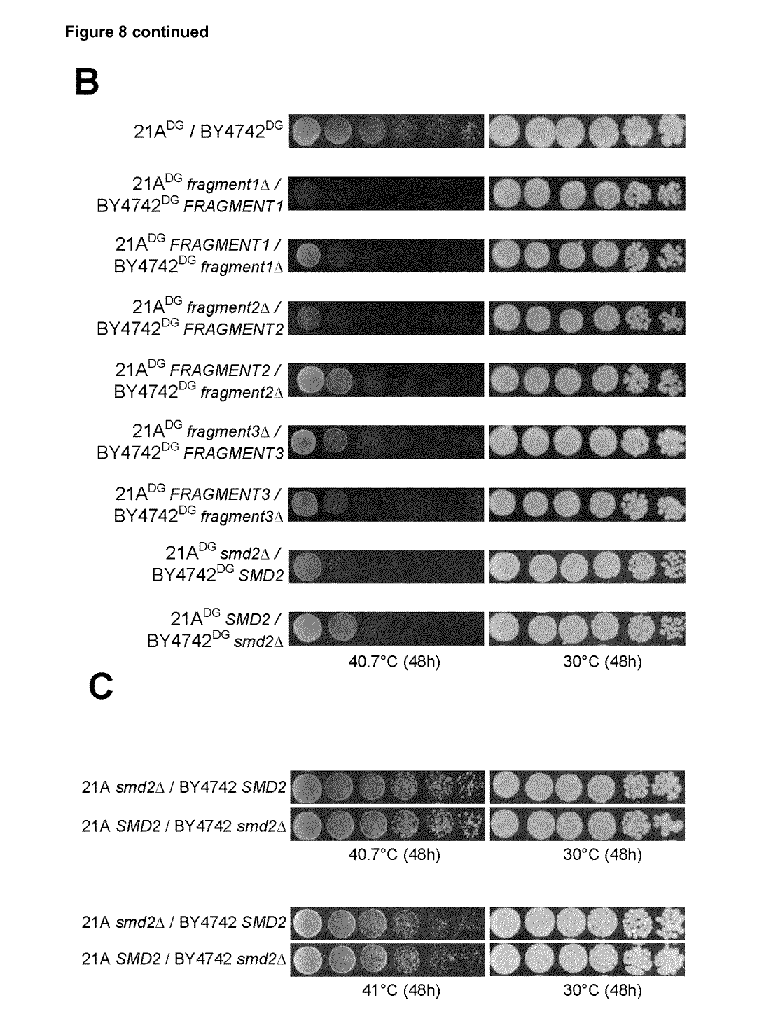

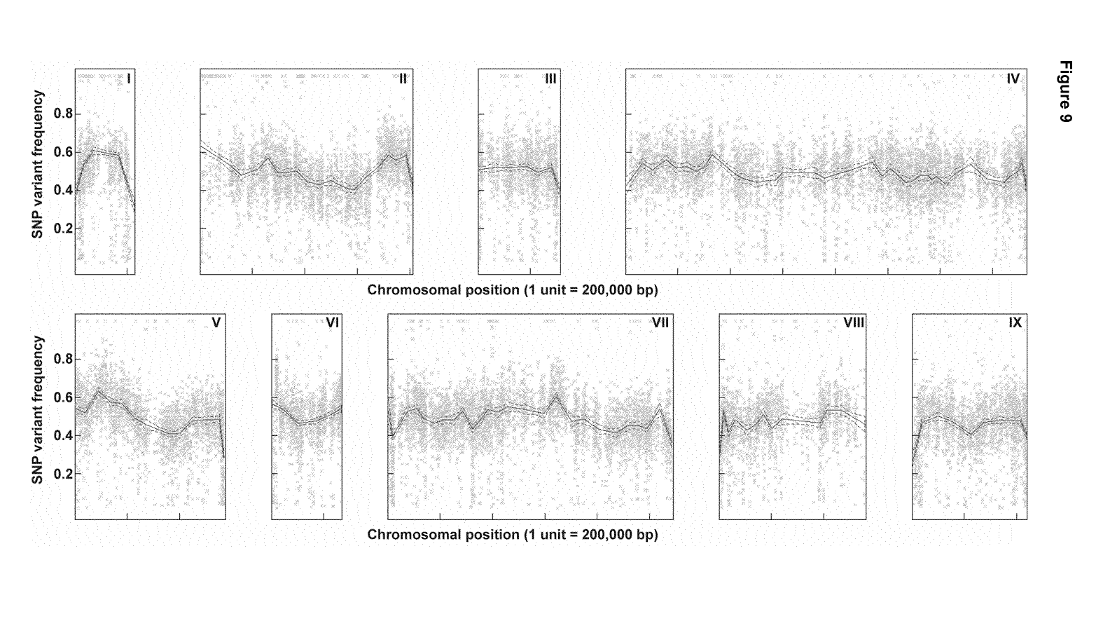

From the over 2200 segregants derived from the diploid 21A^{DG}/BY4742^{DG}, composed of the downgraded parent strains, we selected 58 thermotolerant segregants that grew at 40.7° C. at least as well as the 21A^{DG} superior parent strain. The 58 segregants were separately grown in liquid cultures and pooled based on dry weight for genomic DNA isolation. We performed the same genomic DNA isolation from a pool of 58 unselected segregants. The genomic DNA samples from the selected and unselected pools were sequenced (BGI, Hong Kong) with an average coverage of 37 and 36, respectively, and the sequence reads were aligned to the S288c reference sequence. We have used the same set of SNPs as generated in the previous sequencing of the 21A parent strain compared to S288c, for the mapping of QTLs linked to thermotolerance. The SNP variant frequency was plotted against the chromosomal position for the whole genome (FIG. 2). Interestingly, when we compared the new mapping profile obtained with the segregants of the downgraded parents with the previous mapping profile obtained with the segregants of the original parents, three regions can be discerned with a clear difference (FIG. 2). The previous peak indicating linkage of the region between about 400,000 bp and 600,000 bp on chromosome XIV with the superior parent 21A (QTL1) has shifted to a more upstream position for linkage with the 21A^{DG} downgraded superior parent (QTL8). In the region between 600,000 bp and 800,000 bp on chromosome XII, there is a new conspicuous peak, indicating linkage with the 21A^{DG} superior parent (QTL9). Finally, in the region between 300,000 bp and 500,000 bp on chromosome XVI there is a new peak indicating linkage with the inferior parent BY4742^{DG} (QTL10). There are also less conspicuous changes in other areas. On the other hand, the SNPs within these regions in the unselected pool show 50% association to either parent (FIG. 8). We have scored selected SNPs in the 58 individual thermotolerant segregants and confirmed the statistical significance of the three new QTLs (Table 2). In addition, the significant association of QTL3 with the inferior parent (71%) observed in the first mapping was completely abolished in the second mapping (52%), which confirmed that PRP42 is the only causative gene in this locus.

### Example 7

### Identification of Causative Genes in the New QTLs

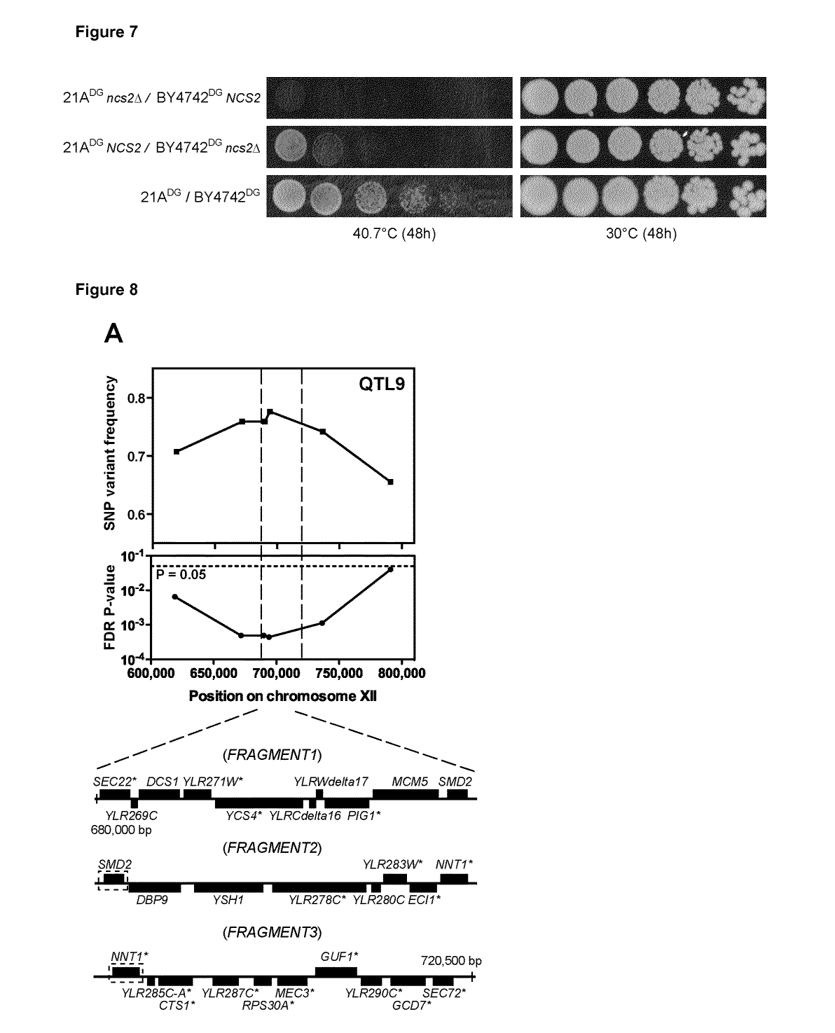

We have focused on QTL8 and QTL9 because they showed the strongest linkage with high thermotolerance (Table 2). In a previous QTL mapping study of thermotolerance (Sinha et al., 2008), the authors identified the NCS2 allele of a clinical isolate as a superior allele compared to the inferior allele from the S288c control strain. Since NCS2 is located in the central region of QTL8 and since the NCS2-21A allele contains the same mutation (A212T) as identified in the previous study, we have tested whether NCS2-21A is also a causative allele in our genetic background. For that purpose, we performed RHA for NCS2 using a hybrid diploid strain constructed from the two downgraded parent strains. We found that the NCS2-21A allele supported higher thermotolerance compared to the NCS2-BY4742 allele, indicating that also in our genetic background the NCS2 allele from the superior strain acted as a causative gene. Deletion of the inferior NCS2-BY4742 allele in the hybrid diploid strain also caused a conspicuous drop in thermotolerance (FIG. 7). Fine-mapping of QTL9 by scoring six selected SNPs individually in all 58 thermotolerant segregants enabled us to reduce the size of the QTL from 150,000 bp to 40,000 bp (FIG. 8A). We then divided this region into three fragments and performed bulk RHA with each fragment in the 21A^{DG}/BY4742^{DG} diploid strain (FIG. 8A). (The fragments had an overlap of one gene.) Evaluation of thermotolerance with the pairs of reciprocally deleted hemizygous strains revealed that FRAGMENT1-21A and FRAGMENT2-21A conferred higher thermotolerance than the corresponding fragments from the inferior BY4742^{DG} parent. For FRAGMENT3 there was no difference (FIG. 8B). We then performed RHA with all individual genes of FRAGMENT1 and 2 containing non-synonymous mutations in their ORF (as indicated in FIG. 8A). However, for none of the genes tested there was a different effect on thermotolerance of the two alleles (data not shown). We then applied RHA to the remaining genes in FRAGMENT2 and found that the SMD2-21A allele conferred higher thermotolerance compared to the SMD2-BY4742 allele (FIG. 8B). Hence, it apparently acted as a causative allele in both FRAGMENT1 and FRAGMENT2, since it was the only gene present in the overlap between the two fragments. The observation that replacement of FRAGMENT1-21A with FRAGMENT1-BY4742 caused a similar reduction in thermotolerance compared to the replacement of FRAGMENT2-21A with FRAGMENT2-BY4742 is consistent with SMD2 being the only causative gene in QTL9.

We performed Sanger sequencing of both SMD2-21A and SMD2-BY4742, which confirmed the Illumina whole-genome sequencing data that nine point mutations were present in the promoter and terminator region of SMD2-21A versus SMD2-BY4742, and that no mutations were present in the ORF. This indicates that the superior character of the SMD2-21A allele is likely due a difference in expression level. Interestingly, the QTL9 region did not show any indication for linkage to the genome of the original superior parent strain 21A, with only 37 out of 58 thermotolerant segregants from 21A/BY4742 having the SMD2-21A allele. We have also applied RHA for SMD2 in the original 21A/BY4742 hybrid and tested thermotolerance at two temperatures (40.7° C. and 41° C.). However, we could not detect any growth difference at both temperatures for the reciprocal hemizygous deletion strains (FIG. 8C). This indicates that our new approach of mapping with the downgraded parent strains is able to reveal minor loci, which completely escape detection with the classical methodologies for genetic mapping and causative gene identification applied for the original parent strains only.

TABLE 1
List of QTLs identified in the mapping with the original parents

| | | Total number | Association | |
|---|---|---|---|---|
| | | of thermo- | to supe- | |
| | | tolerant seg- | rior parent | FDR |
| QTL | Location | regants used | strain 21A | P-value |
| QTL1 | 435069-475213 on | 46 | 100% | 5.16e−13 |
| | chromosome XIV | | | |
| QTL2 | 540838-560167 on | 62 | 74.20% | 1.60e−3 |
| | chromosome II | | | |
| QTL3 | 949927-999889 on | 62 | 29.03% | 1.18e−2 |
| | chromosome IV | | | |
| QTL4 | 645016-659921 on | 62 | 64.52% | 1.36e−1 |
| | chromosome X | | | |
| QTL5 | 595852-651429 on | 62 | 62.90% | 1.69e−1 |
| | chromosome VII | | | |
| QTL6 | 150435-299124 on | 62 | 62.90% | 1.69e−1 |
| | chromosome XII | | | |
| QTL7 | 559076-601738 on | 62 | 61.29% | 2.54e−1 |
| | chromosome XVI | | | |

TABLE 2
List of QTLs identified in the mapping
with the downgraded parents

| | | | Association | |
|---|---|---|---|---|
| | | Total number | to downgrad- | |
| | | of thermo- | ed superior | |
| | | tolerant seg- | parent strain | FDR |
| QTL | Location | regants used | 21A^{DG} | P-value |
| QTL8 | 361531-392591 on | 58 | 91.38% | 1.92e−10 |
| | chromosome XIV | | | |
| QTL9 | 619142-791000 on | 58 | 75.86% | 4.92e−4 |
| | chromosome XII | | | |
| QTL10 | 360037-443597 on | 58 | 31.03% | 9.82e−3 |
| | chromosome XVI | | | |

TABLE 3
Genes previously identified as causative genes in QTLs
with an allele- specific influence on thermotolerance

| | | Function | |
|---|---|---|---|
| Gene | Strain | catergory | Reference |
| MKT1 | YJM789, a thermotolerant | posttranscrip- | (Steinmetz |
| | clinical isolate | tional regulation | et al. 2002) |
| RHO2 | YJM789, a thermotolerant | small GTPase | (Steinmetz |
| | clinical isolate | | et al. 2002) |
| END3 | S96, a descendant of | Endocytosis | (Steinmetz |
| | S288c | | et al. 2002) |
| NCS2 | YJM421, a thermotolerant | tRNA | (Sinha |
| | clinical isolate | modification | et al. 2008) |
| IRA1 | YPS128, a thermotolerant | negative RAS | (Parts |
| | oak tree bark strain | regulator | et al. 2011) |
| IRA2 | YPS128, a thermotolerant | negative RAS | (Parts |
| | oak tree bark strain | regulator | et al. 2011) |
| RSP5 | C3723, a natural | E3 ubiquitin | (Shahsavarani |
| | thermotolerant yeast | ligase | et al. 2011) |
| CDC19 | C3723, a natural | cell division | (Benjaphokee |
| | thermotolerant yeast | cycle, glycolysis | et al. 2012) |

### REFERENCES

Ben-Ari G, Zenvirth D, Shuman A, David L, Klutstein M, Lavi U, Hillel J, Simchen G. 2006. Four linked genes participate in controlling sporulation efficiency in budding yeast. PLoS Genet 2: e195.
Benjamini Y, Yekutieli D. 2005. Quantitative trait Loci analysis using the false discovery rate. Genetics 171: 783-790.
Benjaphokee S, Koedrith P, Auesukaree C, Asvarak T, Sugiyama M, Kaneko Y, Boonchird C, Harashima S. 2012. CDC19 encoding pyruvate kinase is important for high-temperature tolerance in Saccharomyces cerevisiae. N Biotechnol 29: 166-176.
Botstein D, Risch N. 2003. Discovering genotypes underlying human phenotypes: past successes for mendelian disease, future approaches for complex disease. Nat Genet 33 Suppl: 228-237.
Brem R B, Yvert G, Clinton R, Kruglyak L. 2002. Genetic dissection of transcriptional regulation in budding yeast. Science 296: 752-755.
Bullard J H, Mostovoy Y, Dudoit S, Brem R B. 2010. Polygenic and directional regulatory evolution across pathways in Saccharomyces. Proc Natl Acad Sci USA 107: 5058-5063.
Carlborg 0, Haley C S. 2004. Epistasis: too often neglected in complex trait studies? Nat Rev Genet 5: 618-625.
Diezmann S, Dietrich F S. 2011. Oxidative stress survival in a clinical Saccharomyces cerevisiae isolate is influenced by a major quantitative trait nucleotide. Genetics 188: 709-722.
Dimitrov L N, Brem R B, Kruglyak L, Gottschling D E. 2009. Polymorphisms in multiple genes contribute to the spontaneous mitochondrial genome instability of Saccharomyces cerevisiae S288C strains. Genetics 183: 365-383.
Ehrenreich I M, Gerke J P, Kruglyak L. 2009. Genetic dissection of complex traits in yeast: insights from studies of gene expression and other phenotypes in the BYxRM cross. Cold Spring HarbSymp Quant Biol 74: 145-153.
Ehrenreich I M, Torabi N, Jia Y, Kent J, Martis S, Shapiro J A, Gresham D, Caudy A A, Kruglyak L. 2010. Dissection of genetically complex traits with extremely large pools of yeast segregants. Nature 464: 1039-1042.
Gatbonton T, Imbesi M, Nelson M, Akey J M, Ruderfer D M, Kruglyak L, Simon J A, Bedalov A. 2006. Telomere length as a quantitative trait: genome-wide survey and genetic mapping of telomere length-control genes in yeast. PLoS Genet 2: e35.
Goffeau A, Barrell B G, Bussey H, Davis R W, Dujon B, Feldmann H, Galibert F, Hoheisel J D, Jacq C, Johnston M et al., 1996. Life with 6000 genes. Science 274: 546, 563-547.
Hu X H, Wang M H, Tan T, Li J R, Yang H, Leach L, Zhang R M, Luo Z W. 2007. Genetic dissection of ethanol tolerance in the budding yeast Saccharomyces cerevisiae. Genetics 175: 1479-1487.
Johnston J R. 1994. Molecular genetics of yeast: a practical approach. IRL Press at Oxford University Press, Oxford; New York.
Lynch M, Walsh B. 1998. Genetics and analysis of quantitative traits. Sinauer, Sunderland, Mass.
Mancera E, Bourgon R, Brozzi A, Huber W, Steinmetz L M. 2008. High-resolution mapping of meiotic crossovers and non-crossovers in yeast. Nature 454: 479-485.
Marullo P, Aigle M, Bely M, Masneuf-Pomarede I, Durrens P, Dubourdieu D, Yvert G. 2007. Single QTL mapping and nucleotide-level resolution of a physiologic trait in wine Saccharomyces cerevisiae strains. FEMS Yeast Res 7: 941-952.
McLean M R, Rymond B C. 1998. Yeast pre-mRNA splicing requires a pair of U1 snRNP-associated tetratricopeptide repeat proteins. Mol Cell Biol 18: 353-360.
Paterson A H. 1998. Molecular dissection of complex traits. CRC Press, Boca Raton, Fla. Rieseberg L H, Archer M A, Wayne R K. 1999. Transgressive segregation, adaptation and speciation. Heredity (Edinb) 83 (Pt 4): 363-372.
Risch N J. 2000. Searching for genetic determinants in the new millennium. Nature 405: 847-856.
Shepherd A, Piper P W. 2010. The Fps1p aquaglyceroporin facilitates the use of small aliphatic amides as a nitrogen source by amidase-expressing yeasts. FEMS Yeast Res 10: 527-534.
Sherman F, Hicks J. 1991. Micromanipulation and dissection of asci. Methods Enzymol 194: 21-37.
Sinha H, David L, Pascon R C, Clauder-Munster S, Krishnakumar S, Nguyen M, Shi G, Dean J, Davis R W, Oefner P J et al., 2008. Sequential elimination of major-effect contributors identifies additional quantitative trait loci conditioning high-temperature growth in yeast. Genetics 180: 1661-1670.
Smith E N, Kruglyak L. 2008. Gene-environment interaction in yeast gene expression. PLoS Biol 6: e83.
Steinmetz L M, Sinha H, Richards D R, Spiegelman J I, Oefner P J, McCusker J H, Davis R W. 2002. Dissecting the architecture of a quantitative trait locus in yeast. Nature 416: 326-330.
Swinnen S, Schaerlaekens K, Pais T, Claesen J, Hubmann G, Yang Y, Demeke M, Foulquie-Moreno M R, Goovaerts A, Souvereyns K et al., 2012. Identification of novel causative genes determining the complex trait of high ethanol tolerance in yeast using pooled-segregant whole-genome sequence analysis. Genome Res.
Wach A, Brachat A, Pohlmann R, Philippsen P. 1994. New heterologous modules for classical or PCR-based gene disruptions in Saccharomyces cerevisiae. Yeast 10: 1793-1808.
Winzeler E A, Shoemaker D D, Astromoff A, Liang H, Anderson K, Andre B, Bangham R, Benito R, Boeke J D, Bussey H et al., 1999. Functional characterization of the S. cerevisiae genome by gene deletion and parallel analysis. Science 285: 901-906.

1. A method of inducing thermotolerance in a yeast, the method comprising:
utilizing a small nuclear ribonucleoprotein particle protein to obtain thermotolerance in the yeast.

2. The method according to claim 1, wherein said small nuclear ribonucleoprotein particle protein is a Prp42 protein and/or a Smd2 protein.

3. The method according to claim 1, wherein said small nuclear ribonucleoprotein particle protein comprises SEQ ID NO: 2 or SEQ ID NO: 4.

4. The method according to claim 1, wherein said yeast is a Saccharomyces sp.

5. A recombinant yeast, comprising a recombinant gene encoding a protein comprising SEQ ID NO: 2 and/or SEQ ID NO: 4.

6. The recombinant yeast according to claim 5, comprising SEQ ID NO:1 or SEQ ID NO: 2.

7. The recombinant yeast according to claim 5, wherein said yeast is a Saccharomyces sp.

8. A method to obtain a thermotolerant yeast, the method comprising:
crossing two parental yeast strains, wherein at least one parental strain encodes a protein comprising SEQ ID NO:2 and/or SEQ ID NO: 4.

9. A method to obtain a thermotolerant yeast, the method comprising: transforming a yeast with a gene encoding a protein comprising SEQ ID NO: 2 and/or SEQ ID NO: 4.

10. A method for screening for thermotolerance in yeast, the method comprising:
(1) identifying at least one gene responsible for thermotolerance;
(2) downgrading said at least one gene in a yeast strain;
(3) crossing two downgraded yeast strains to produce a cross; and
(4) screening for thermotolerance genes in the offspring of said cross.

11. The method according to claim 2, wherein the small nuclear ribonucleoprotein particle protein comprises SEQ ID NO: 2 or SEQ ID NO: 4.

12. The method according to claim 2, wherein the yeast is a Saccharomyces sp.

13. The method according to claim 3, wherein the yeast is a Saccharomyces sp.

14. The method according to claim 11, wherein the yeast is a Saccharomyces sp.

15. The recombinant yeast of claim 6, wherein the yeast is a Saccharomyces sp.

16. A Saccharomyces sp. comprising an exogenous polynucleotide encoding a peptide comprising SEQ ID NO: 2 and/or SEQ ID NO: 4, wherein the Saccharomyces sp. is thermotolerant in comparison to a Saccharomyces sp. without the exogenous polynucleotide.

17. The Saccharomyces sp. of claim 16, wherein the exogenous polynucleotide encodes a peptide comprising SEQ ID NO: 2 and SEQ ID NO: 4.

18. The Saccharomyces sp. of claim 16, wherein the exogenous polynucleotide encodes a peptide comprising SEQ ID NO: 2.

19. The Saccharomyces sp. of claim 16, wherein the exogenous polynucleotide encodes a peptide comprising SEQ ID NO: 4.


SEQUENCE LISTING (4 sequences)

SEQ ID NO: 1 - DNA, 1635 bp, Saccharomyces cerevisiae
  CDS (1)..(1635)
atggataaatatactgctttgattcacgatgagaatttttcaactcttacattgaatgtctcgagataccctaaaagtttggcatattgggagaagttactgaactatatagtaaaggcttcggcaccaatatgcaagtccactgagcctcaattattaaagttaatacgatgtacttattcttctatgctgaatgagtttccctatctagaaaactactatatcgacttcgcgctactggaatacaaactagggaatgtttctatgtctcataagatatttcagcgcggattgcaagcctttaaccaaagatctttactattatggacttcatatttaaagttttgtaataacgttatctcacaccagaagcagttgtttaaaaaatatgaaacagctgaagaatatgttggtttgcattttttcagtggggaattctgggatttatacctggaacaaattagctcaagatgtacgtcttctaagaaatattggaacgttttgaggaagatactcgaaattcctttacactctttttcgaaattttatgcattatggttacagcgcatagacgatataatggacctaaagcaattatcacaactaacaagtaaagatgaattattgaagaaactcaagatagacatcaattacagtggaagaaagggaccgtacctgcaggatgcaaagaaaaagttgaagaaaataacgaaggaaatgtacatggttgtacaatatcaagtacttgaaatctattccatttttgaatcaaaaatttatatcaactattatacatcgccagaaaccttggtttcttcagatgaaatagaaacatggataaaatacttggattataccatcaccttacaaacagattctttaacacatttaaattttcaaagagctttacttcctctggcccattatgatcttgtatggataaaatacagtaaatggttgataaattcaaaaaatgaccttttgggggcaaaaaacgttctattaatgggtcttaaattttctttgaagaagaccgaaataataaaactactgtattcagttatttgcaagttaaatgagtacgtacttttgcgaaatctattggaaaaaattgagtcctcatattccgacaatgttgaaaacgttgatgattttgagatattttgggactatcttcagttcaaaacgttctgccagaattctctgtattccagtcgatattctgattcacaatcaaacgggcttttgaataaggagctgtttgataaagtatggaaacgtttgagttgtaaagaaaaaaaaagtggtcaagaaattttattgaataacttagtacagttttattcgaaggataccgtagaatttgtagaaaagaacatatttcaaaagataatagaatttggttgggagtactacctacagaacggtatgttctggaactgttattgccgtttaatttatttcgacacttctaggtcatatttggacaagaggcaatatattgtcagaaaaatatggccccaaatagacaaaaaatttgcacagagcgtcttgccgtctttaacagaattctgtgagtcatacttcccagaggagatggatacattagaggaaatgtttactgaagaaccttag

SEQ ID NO: 2 - protein, 544 aa, Saccharomyces cerevisiae
MDKYTALIHDENFSTLTLNVSRYPKSLAYWEKLLNYIVKASAPICKSTEPQLLKLIRCTYSSMLNEFPYLENYYIDFALLEYKLGNVSMSHKIFQRGLQAFNQRSLLLWTSYLKFCNNVISHQKQLFKKYETAEEYVGLHFFSGEFWDLYLEQISSRCTSSKKYWNVLRKILEIPLHSFSKFYALWLQRIDDIMDLKQLSQLTSKDELLKKLKIDINYSGRKGPYLQDAKKKLKKITKEMYMVVQYQVLEIYSIFESKIYINYYTSPETLVSSDEIETWIKYLDYTITLQTDSLTHLNFQRALLPLAHYDLVWIKYSKWLINSKNDLLGAKNVLLMGLKFSLKKTEIIKLLYSVICKLNEYVLLRNLLEKIESSYSDNVENVDDFEIFWDYLQFKTFCQNSLYSSRYSDSQSNGLLNKELFDKVWKRLSCKEKKSGQEILLNNLVQFYSKDTVEFVEKNIFQKIIEFGWEYYLQNGMFWNCYCRLIYFDTSRSYLDKRQYIVRKIWPQIDKKFAQSVLPSLTEFCESYFPEEMDTLEEMFTEEP

SEQ ID NO: 3 - DNA, 1164 bp, Saccharomyces cerevisiae
caacaataaagtcttagcttggcgccttttctacttgtttgcatgtaatgaatctaaaaaattcgctacaaatacttcattattatatttctatataaattctgtaaacgaaattatactttttgaggtttcatctgcgtctcatattctttttgtataataaacgcacagacttgatatgaccgtcaatcaaaataattcaaagcttcttaaaaaagaaattgaaatcaaagacatacgaaagaaattagaaaatagaggtcgatttaccactgaacaatcctacaatacatgagctcttgaagtgactttatggggaccaatttgttctatcgtgatattactacgggtaatactaccaaatgaaaattatcttactcaaaaggtatgacaagaacatcatcactagaacatcatcattcttaatagcaagcgtcatcacgtttgccccgcctttgacagttgattagaggagtattataaaaccgttttattatgtcgtatgtttgatcttaaccatttttacatcctataatttggagtgaatttttatatactaacttaaaagtaattgatttgtatctttccagttcacaaataattgatcgtccaaaacatgaactctctagagcagaattagaggaactagaagaatttgaattcaaacatggtccaatgtccctgataaatgatgctatggtgacaagaacacctgtgataatctcattaagaaacaatcataaaataatagcgagagtgaaagctttcgacaggcattgtaatatggttttagaaaatgtgaaggagctttggacagagaagaagggcaaaaatgtaattaatcgggaaagattcataagtaaactattcttaagaggtgattcagttatcgttgtgttaaaaacccctgttgagtaaaaagtaagacaagcagccctcaatctacaaggaaagcaaaccatcgtaagaaactttacagtcagcggaaagcctttttttattttggaagtaagaccaattattattggctgactttctagctcttgcgaaagtattgtattcaagaaagcgaataaagcatctatattatcacatttaattaaagagcatttttaataagtagatttttacataaaatatagaataaatatcaatattattaat

SEQ ID NO: 4 - protein, 110 aa, Saccharomyces cerevisiae
MSSQIIDRPKHELSRAELEELEEFEFKHGPMSLINDAMVTRTPVIISLRNNHKIIARVKAFDRHCNMVLENVKELWTEKKGKNVINRERFISKLFLRGDSVIVVLKTPVE